(19) United States
(12) Patent Application Publication
(10) Pub. No.: US 2024/0091318 A1
(43) Pub. Date: Mar. 21, 2024

(54) COMBINATION THERAPY COMPRISING LONG ACTING GLP-1/GLUCAGON AND NPY2 RECEPTOR AGONISTS

(71) Applicant: Boehringer Ingelheim International GmbH, Ingelheim am Rhein (DE)

(72) Inventors: Robert AUGUSTIN, Biberach (DE); Anouk OLDENBURGER, Mittelbiberach (DE)

(21) Appl. No.: 18/450,487

(22) Filed: Aug. 16, 2023

(30) Foreign Application Priority Data
Aug. 18, 2022 (EP) 22191099.5

(51) Int. Cl. A61K 38/26; A61K 38/22; A61P 3/04

(52) U.S. Cl. CPC A61K 38/26; A61K 38/2271; A61P 3/04

(57) ABSTRACT

A combination therapy is provided, which comprises
Compound I (GLP-1R/glucagon receptor dual agonist): H-His-Ac4c-Gln-Gly-Thr-Phe-Thr-Ser-Asp-Tyr-Ser-Lys-Tyr-Leu-Asp-Glu-Arg-Ala-Ala-Lys-Asp-Phe-Ile-Lys(HOOC—(CH₂)₁₆-CO-γGlu-Gly-Ser-Gly-Ser-Gly-Gly-)-Trp-Leu-Glu-Ser-Ala-NH₂ (I) and
a long acting NPY2 receptor agonist selected from Compound A to L.

The combination therapy might be useful to treat obesity and various obesity-related conditions, diseases, or co-morbidities, such as type 2 diabetes, liver diseases such as NAFLD (non-alcoholic fatty liver disease) and NASH (non-alcoholic steatohepatitis), kidney diseases, or cardiovascular diseases.

### RELATED APPLICATIONS

This application is a nonprovisional patent application which claims priority under 35 U.S.C. 119(b) and 37 CFR 1.55 to pending EP Serial No. EP 22 191 099.5, filed Aug. 18, 2022, which is incorporated herein by reference in its entirety.

### SEQUENCE DISCLOSURE

This application includes, as part of its disclosure, a “Sequence Listing XML” pursuant to 37 C.F.R. § 1.831(a) which is submitted in XML file format via the USPTO patent electronic filing system in a file named “01-3536-US-1-2023-08-16-SL.xml”, created on Aug. 11, 2023, and having a size of 35,799 bytes, which is hereby incorporated by reference herein in its entirety.

### BACKGROUND OF THE INVENTION

### Field of the Invention

The present invention relates to the pharmacology and biology for a combination of long-acting, acylated PYY analogues that are neuropeptide Y2 (NPY2) receptor agonists with dual glucagon-like peptide-1 (GLP-1)/glucagon (GCG) receptor agonists and their medical use in the treatment and/or prevention of a variety of diseases, conditions or disorders, such as treatment and/or prevention of excess food intake, excess body weight, obesity, metabolic diseases, and other conditions or disorders related to excess body weight or obesity, e.g. diabetes, kidney disease, non-alcoholic-steatohepatitis, and cardiovascular diseases.

### Obesity

Obesity is a chronic, relapsing, progressive, disease and represents one of greatest healthcare challenges of our times. In 2016, more than 1.9 billion adults aged 18 years and older were overweight and of these over 650 million adults were obese (BMI≥30 kg/m²). Despite long-standing efforts, the number of overweight and obese patients is still growing with a prevalence of obesity that nearly tripled between 1975 and 2016. This equates to 39% (39% of men and 40% of women) of adults aged 18 or over who were overweight, with 13% obese.

Overweight and obesity are defined as abnormal or excessive fat accumulation that presents a risk to health. In this regard, overweight and obesity are major risk factors for a number of chronic diseases which are directly (co-morbidities) and indirectly (complications) associated that include but are not limited to cardiovascular diseases (e.g. heart failure), cardiometabolic diseases such as insulin resistance, type 2 diabetes, atherosclerosis, cardiovascular diseases, hypertension, dyslipidaemia, hyperuricemia, chronic kidney disease, non-alcoholic fatty liver disease (NAFLD), non-alcoholic steatohepatitis (NASH), liver cirrhosis, depression, sleep apnoea, musculoskeletal disorders (e.g. osteoarthritis), gall bladder disease, and certain types of cancer. In the US, obesity is now believed to be the second-leading cause of preventable death after smoking.

If current trends continue, it is estimated that 2.7 billion adults will be overweight, over 1 billion affected by obesity, and 177 million adults severely affected by obesity by 2025. The rise in obesity drives an increase in diabetes, and approximately 90% of people with type 2 diabetes may be classified as obese. There are 246 million people worldwide with diabetes, and by 2025 it is estimated that 380 million will have diabetes. First line therapy for overweight and obese patients comprises diet and exercise but often are not sufficiently efficacious. Second line treatment options are bariatric surgery and pharmacotherapy. Available pharmacological treatments seem to lack in efficacy and/or safety, and only a limited number of approved therapies are available in the US and in Europe. Therefore, there is still a high medical need for more efficacious and safe treatment options.

### Glucagon, GLP-1 and Oxyntomodulin

Pre-proglucagon is a 158 amino acid precursor polypeptide that is differentially processed in the tissues to form a number of structurally related proglucagon-derived peptides, including glucagon (Glu), glucagon-like peptide-1 (GLP-1), glucagon-like peptide-2 (GLP-2), and oxyntomodulin (OXM). These molecules are involved in a wide variety of physio-logical functions, including glucose homeostasis, insulin secretion, gastric emptying and intestinal growth, as well as regulation of food intake. Glucagon is a 29-amino acid peptide that corresponds to amino acids 53 to 81 of pre-proglucagon. Oxyntomodulin (OXM) is a 37 amino acid peptide which includes the complete 29 amino acid sequence of glucagon with an octapeptide carboxyterminal extension (amino acids 82 to 89 of pre-proglucagon). The major biologically active fragment of GLP-1 is produced as a 30-amino acid, C-terminally amidated peptide that corresponds to amino acids 98 to 127 of pre-proglucagon. GLP-1 decreases elevated blood glucose levels by improving glucose-stimulated insulin secretion and promotes weight loss chiefly through decreasing food intake. Clinically, GLP-1 receptor agonists are established therapies for the treatment of Diabetes and Obesity with a therapeutic window that is limited due to their mechanism-related gastrointestinal side-effect profile (e.g. nausea and vomiting). Glucagon helps maintain the level of glucose in the blood by binding to glucagon receptors on hepatocytes, causing the liver to release glucose—stored in the form of glycogen—through glycogenolysis. As these stores become depleted, glucagon stimulates the liver to synthesize additional glucose by gluconeo-genesis. Glucagon has been demonstrated preclinically and clinically to affect body weight by increasing energy expenditure. This glucose is released into the bloodstream, preventing the development of hypoglycemia. OXM is released into the blood in response to food ingestion and in proportion to meal calorie content. OXM activates both the glucagon and GLP-1 receptors, with a slightly higher potency for the glucagon receptor over the GLP-1 receptor but is less potent than native glucagon and GLP-1 on their respective receptors. Human glucagon is also capable of activating both receptors, though with a strong preference for the glucagon receptor over the GLP-1 receptor. GLP-1 on the other hand is not capable of activating glucagon receptors. OXM is involved in regulation of body weight and has been shown to suppress appetite and inhibit food intake in humans as well as energy expenditure.

Other peptides have been stated to bind to and activate both the glucagon receptor and the GLP-1 receptor and to suppress body weight gain (see, e.g. WO2011/075393, WO2014/041195, WO2015/183054, WO2016/065090, WO2016/108617, WO2017/074798)

### PYY

Peptide YY (PYY) is a peptide consisting of 36 amino acid is secreted from endocrine cells (L cells) of the gastrointestinal tract along with diet ingestion and exhibits a feeding suppressive action via Y2 receptors (Inhibition of Food Intake in Obese Subjects by Peptide YY₃₋₃₆, N Engl J Med 2003; 349; 941-8). The Intestine/hypothalamus pathway via Y2 receptors of hypothalamic arcuate nucleus NPY/AgRP-expressing neurons, and the vagal afferent pathway via Y2 receptors of vagal nerve ending have been reported as its anorexic mechanism of action, which for native PYY(3-36) is associated with limited tolerability in human due to dose-dependent nausea and vomiting when administered intranasally. PYY is cleaved to PYY(3-36) by dipeptidyl peptidase IV (DPP IV). PYY(3-36) displays increased selectivity for the neuropeptide Y2 receptor over neuropeptide Y1, Y4 and Y5 receptors as compared to PYY(1-36), albeit some Y1 and Y5 affinity is retained. However, PYY and also PYY(3-36) have a short half-life in the body and show undesirable chemical or physical properties, e.g. low stability. Further, the pharmacologic effect, e.g. its efficacy as body weight lowering agent, seems limited, however, long-acting analogues of PYY3-36 have been demonstrated preclinically and clinically to provide a therapeutic window achieving body weight lowering efficacy with an acceptable tolerability profile.

WO2014/178018 discloses PYY analogues and their ability to reduce food intake in mice. WO2011/033068 and WO2011/058165 disclose long acting NPY2R agonists. WO2015/071355, WO2016/198682 and WO2020/092191 relate to PYY compounds, which are selective NPY2R agonists. PYY compounds are disclosed comprising a covalently attached substituent or modifying group also referred therein as a protracting moiety.

### GLP-1 and NPY2 Receptor Agonism

Simultaneous activation of the GLP-1R and NPY2R, as well as synergistic food intake reduction and body weight lowering efficacy has been demonstrated in preclinical species, e.g. for mice (Neary N M et al. Endocrinology 2005; 146:5120; Talsania T et al. Endocrinology 2005; 146: 3748; Kjargaard M. et al. Neuropeptides 2019; 73:89; WO2011/039096; WO2019/207505), rats (Reidelberger R D et al. Obesity 2011; 19: 121; Dischinger U et al. Frontiers in Endocrinology 2021; 11:598843; WO2017/035432), and pigs (WO2015/071355).

In humans, synergy in reducing energy intake was demonstrated with the administration (continuous infusion) of GLP-1 together with PYY3-36 (Schmidt J B et al. Am J Physiol Endocrinol Metab 2014; 306: E1248) or of PYY3-36, GLP-1 and Oxyntomodulin (Field BCT et al. Diabetes 2010 59:1635; Tan T et al. J. Clin. Endocrinol Metab 2017; 102: 2364; Tan T et al., Diabetes Care 2019, 42(8):1446)

### BRIEF SUMMARY OF THE INVENTION

The present invention provides a combination therapy that comprises administration of a long acting GLP-1R/GCGR dual agonist and a long acting NPY2R agonist. The simultaneous activation of the GLP-1R, GCGR and NPY2 receptor has the potential to provide a very effective treatment method to reduce food intake, reduce appetite and/or reduce body weight by a simultaneous, synergistic reduction in energy intake through GLP-1R and NPY2R agonism and an increase in energy expenditure via GCGR activation. According to the present invention, it is possible to activate the three receptors by long-acting agents following administration by subcutaneous injections. Further, the combination therapy might efficaciously be administered by once weekly injection(s) of the agonists.

The selection of the NPY2R agonist, which is to be combined with the GLP-1R/GCGR dual agonist, seems to be important. However, selecting efficacious NPY2R agonists is not predicted or taught e.g., obvious in light of the data provided in the prior art. (e.g. WO2021/094259 or WO2022/029231).

Further, the combination therapy according to the present invention herewith seems to provide a longer lasting effect on food intake inhibition when compared to a combination of a GLP-1R agonist with a NPY2R agonist or when compared to the GLP-1R/GCGR alone. The invention relates to a combination therapy comprising administering a long-acting GLP-1/glucagon receptor dual agonist and a long acting NPY2 receptor agonist.

More specifically, the invention relates to a combination therapy comprising administering to a patient
an effective, and/or therapeutically effective, amount of Compound I:

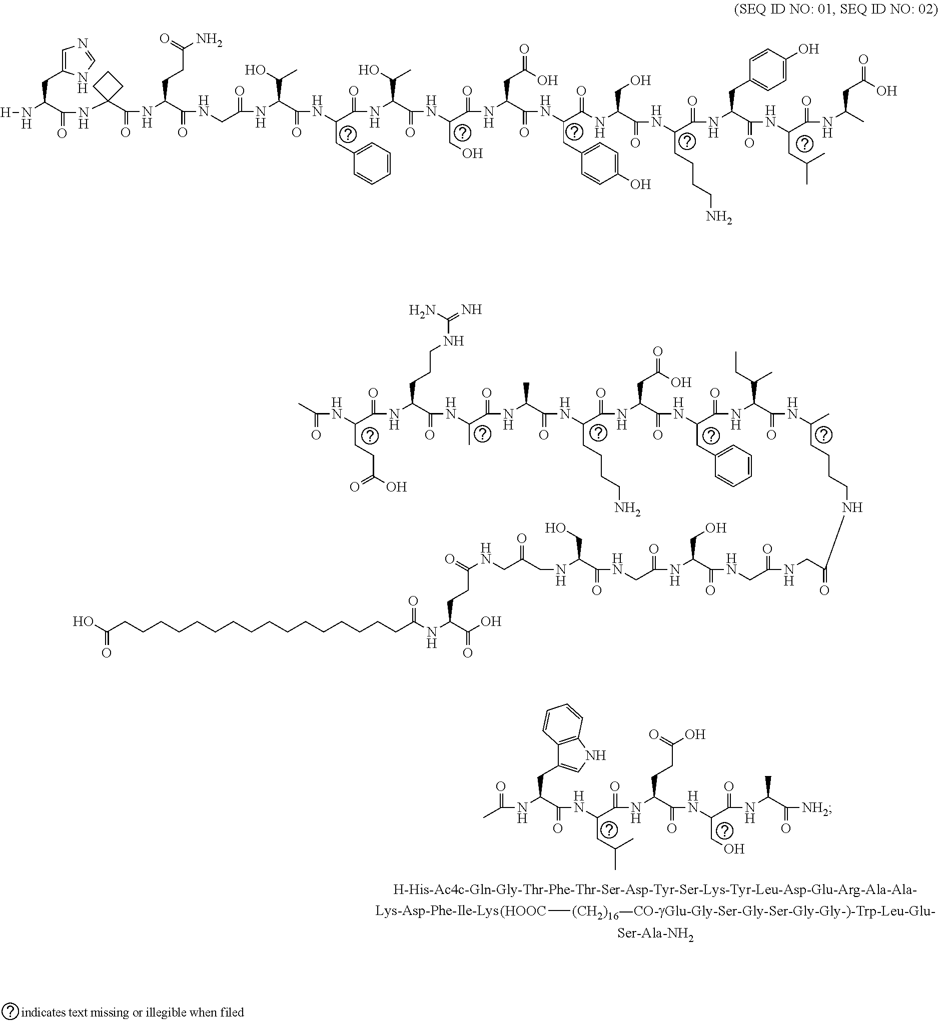

and
a long acting NPY2 receptor agonist selected from the group consisting of Compound A to L.

In an embodiment, the long acting NPY2 receptor agonist is selected from the group consisting of Compound B, E, K, and L.

In an embodiment, the weight ratio between Compound I and the NPY2 receptor agonist is in the range of 1:5 to 5:1 (Compound I: NPY2 receptor agonist).

Use of a PYY analogue selected from the group consisting of Compound A to L in a combination therapy together with Compound I.

The compounds may be administered once per week (once weekly) or more often.

In an embodiment, Compound I and the long acting NPY2 receptor agonist is administered via subcutaneous injection, e.g., via two separate injections or via one injection administering both compounds simultaneously. In case of two separate injections, one of the compounds may be administered first immediately followed by the other one, or there might be a time period in between the two injections, such as 4, 3, 2 or 1 day. Alternatively, the time in between the injections is 12, 6, 4, 2 or 1 hour.

### BRIEF DESCRIPTION OF THE FIGURES

FIG. 1: Effect of Compound B on (A) food intake (gram) and (B) relative (percent) body weight in DIO mice. DIO animals were administrated with different doses of Compound B once daily. Relative body weight was measured in % to baseline body eight of each animal per day for a period of 28 days. Food intake measured in gram per animal per day for a period of 28 days. Data presented as mean±SEM. * p<0.05, ** p<0.01, *** p<0.001; One-Way ANOVA comparison to vehicle group followed by Dunnett's multiple comparison testing.

FIG. 2: Effect of Compound E on (A) food intake (gram) and (B) relative (percent) body weight in DIO mice. DIO animals were administrated with different doses of Compound E once daily. Relative body weight was measured in % to baseline body weight of each animal per day for a period of 28 days. Food intake measured in gram per animal per day for a 5 period of 28 days. Data presented as mean±SEM. * p<0.001; One-Way ANOVA comparison to vehicle group followed by Dunnett's multiple comparison testing.

FIG. 3: Effect of Compound K on (A) food intake (gram) and (B) relative (percent) body weight in DIO mice. DIO animals were administrated with different doses of Compound K once daily. Relative body weight was measured in % to baseline body eight of each animal per day for a period of 8 days. Food intake measured in gram per animal per day for a period of 8 days. Data presented as mean±SEM. * p<0.05, ** p<0.01, *** p<0.001; One-Way ANOVA comparison to vehicle group followed by Dunnett's multiple comparison testing.

FIG. 4: Effect of Compound L on (A) food intake (gram) and (B) relative (percent) body weight in DIO mice. DIO animals were administrated with different doses of Compound L once daily. Relative body weight was measured in % to baseline body eight of each animal per day for a period of 8 days. Food intake measured in gram per animal per day for a period of 8 days. Data presented as mean±SEM. * p<0.05, ** p<0.01, *** p<0.001; One-Way ANOVA comparison to vehicle group followed by Dunnett's multiple comparison testing.

FIG. 5: Effect of Compound B alone or in combination with Semaglutide on (A) relative (percent) body weight, (B) food intake (gram), (C) cumulative food intake (gram) and (D) area under the curve of graph B in DIO mice. DIO animals were administrated with different doses of Compound B alone or in combination with Semaglutide once daily. Relative body weight was measured in % to baseline body weight of each animal per day for a period of 28 days. Food intake measured in gram per animal per day for a period of 12 days. Data presented as mean±SEM. * p<0.05, ** p<0.01, *** p<0.001, **** p<0.0001; One-Way ANOVA comparison to Semaglutide group followed by Dunnett's multiple comparison testing.

FIG. 6: Effect of Compound B alone or in combination with Compound I on (A) relative (percent) body weight, (B) food intake (gram), (C) cumulative food intake (gram) and (D) area under the curve of graph B in DIO mice. DIO animals were administrated with different doses of Compound B alone or in combination with compound I once daily. Relative body weight was measured in % to baseline body weight of each animal per day for a period of 28 days. Food intake measured in gram per animal per day for a period of 11 days. Data presented as mean±SEM. * p<0.05, ** p<0.01, *** p<0.001, **** p<0.0001; One-Way ANOVA comparison to Compound I group followed by Dunnett's multiple comparison testing.

FIG. 7: Effect of Compound E alone or in combination with Semaglutide on (A) relative (percent) body weight, (B) food intake (gram), (C) cumulative food intake (gram) and (D) area under the curve of graph B in DIO mice. DIO animals were administrated with different doses of Compound E alone or in combination with Semaglutide once daily. Relative body weight was measured in % to baseline body weight of each animal per day for a period of 28 days. Food intake measured in gram per animal per day for a period of 12 days. Data presented as mean±SEM. * p<0.05, ** p<0.01, *** p<0.001, **** p<0.0001; One-Way ANOVA comparison to Semaglutide group followed by Dunnett's multiple comparison testing.

FIG. 8: Effect of Compound E alone or in combination with Compound I on (A) relative (percent) body weight, (B) food intake (gram), (C) cumulative food intake (gram) and (D) area under the curve of graph B in DIO mice. DIO animals were administrated with different doses of Compound E alone or in combination with compound I once daily. Relative body weight was measured in % to baseline body weight of each animal per day for a period of 28 days. Food intake measured in gram per animal per day for a period of 11 days. Data presented as mean±SEM. * p<0.05, ** p<0.01, *** p<0.001, **** p<0.0001; One-Way ANOVA comparison to Compound I group followed by Dunnett's multiple comparison testing.

FIG. 9: Effect of Compound K alone or in combination with Semaglutide on (A) relative (percent) body weight, (B) food intake (gram), (C) cumulative food intake (gram) and (D) area under the curve of graph B in DIO mice. DIO animals were administrated with different doses of Compound K once daily. Relative body weight was measured in % to baseline body weight of each animal per day for a period of 28 days. Food intake measured in gram per animal per day for a period of 12 days. Data presented as mean±SEM. * p<0.05, ** p<0.01, *** p<0.001, **** p<0.0001; One-Way ANOVA comparison to Semaglutide group followed by Dunnett's multiple comparison testing.

FIG. 10: Effect of Compound K alone or in combination with Compound I on (A) relative (percent) body weight, (B) food intake (gram), (C) cumulative food intake (gram) and (D) area under the curve of graph B in DIO mice. DIO animals were administrated with different doses of Compound K alone or in combination with compound I once daily. Relative body weight was measured in % to baseline body weight of each animal per day for a period of 28 days. Food intake measured in gram per animal per day for a period of 12 days. Data presented as mean±SEM. * p<0.05, ** p<0.01, *** p<0.001, **** p<0.0001; One-Way ANOVA comparison to Compound I group followed by Dunnett's multiple comparison testing.

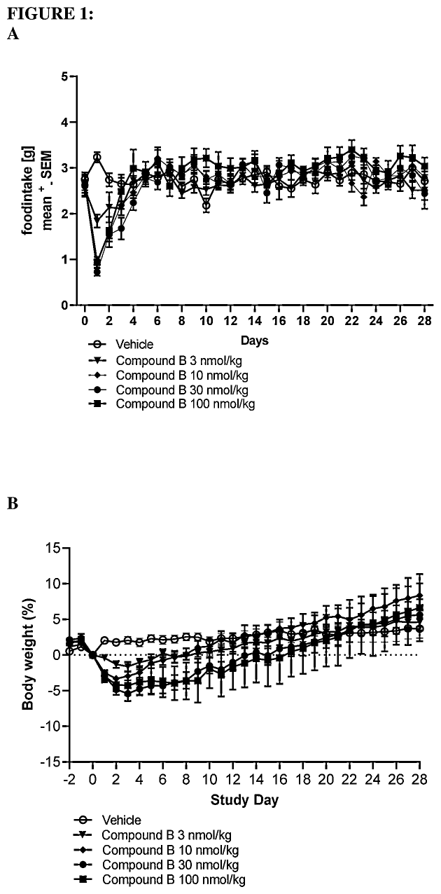

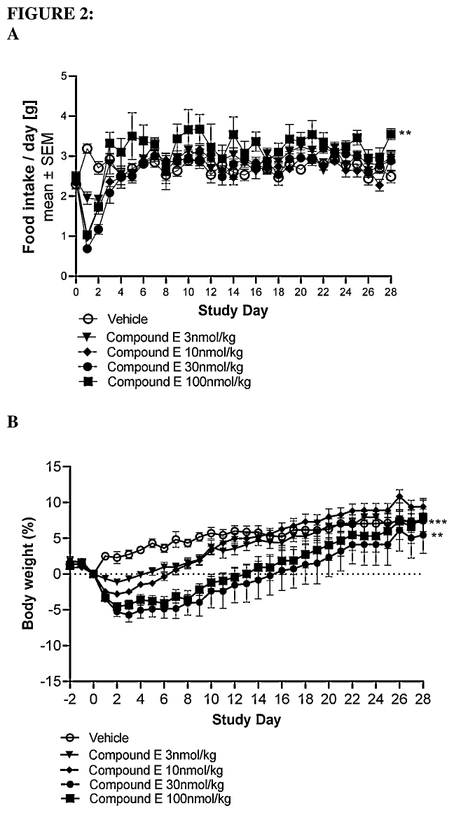

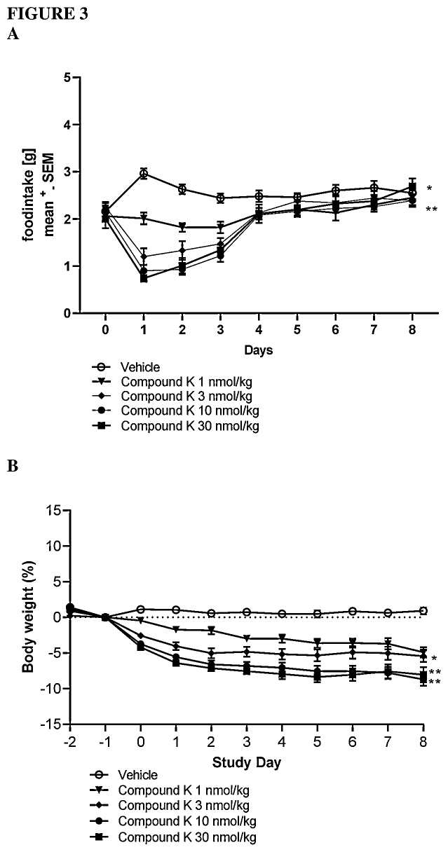

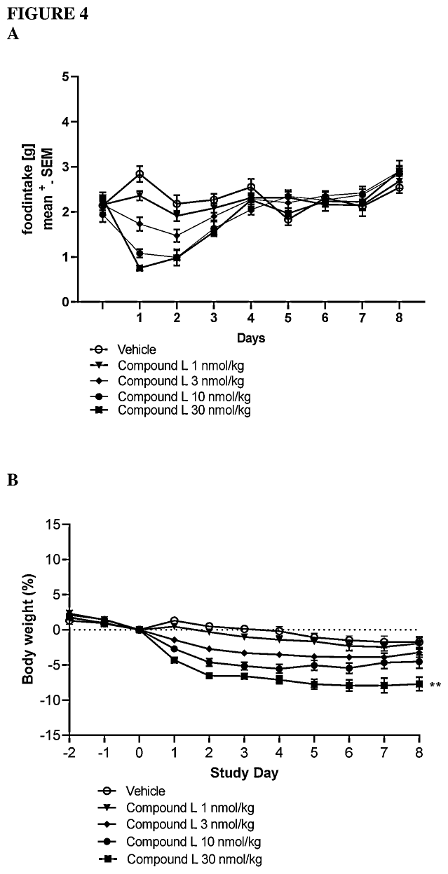

### DETAILED DESCRIPTION OF THE INVENTION

### Additional Abbreviations

gGlu or γGlu: L-γ-glutamyl
iVal: 3-methylbutanoyl (isovalerianoyl)
C18DA: 17-carboxy-heptadecanoyl (HOOC—(CH₂)₁₆—CO—)

The synergistic improvement in treatment outcome may be a greater than additive improvement in overall efficacy as measured by one or more of the following: reduction of food intake, reduction of appetite and/or reduction of body weight obtained by co-administration as compared with administration of either agent alone.

“Combination therapy” in general refers to administration of two or more active ingredients simultaneously or sequentially, such that the concentration of the individual active ingredients in the body is high enough to exhibit a synergistic effect. Combination therapy according to the invention can occur with or without instructions for combined use. The two active ingredients may thus be administered entirely separately or be entirely separate pharmaceutical dosage forms. The individual active ingredients may be pharmaceutical compositions that are also sold independently of each other and where just instructions for their combined use are provided in the package equipment, e.g. leaflet or the like, or in other information e.g. provided to physicians and medical staff (e.g. oral communications, communications in writing or the like), for simultaneous or sequential use for being jointly active. It can refer to either a fixed combination in one dosage unit form, or a kit of parts for the combined administration where Compound I and a Compound selected from the group consisting of Compound A to L (or semaglutide and a Compound selected from the group consisting of Compound A to L) may be administered independently at the same time or separately within time intervals, especially where these time intervals allow that the combination partners show a cooperative (synergistic) effect.

The terms “co-administration” or “combined administration” or the like as utilized herein are meant to encompass administration of the individual active ingredients to a single subject in need thereof (e.g. a patient), and are intended to include treatment regimens, in which the agents are not necessarily administered by the same route of administration and/or at the same time.

The term “fixed combination” means that the active ingredients, e.g. Compound I and a Compound selected from the group consisting of Compound A to L (or semaglutide and a Compound selected from the group consisting of Compound A to L) are both administered to a patient simultaneously in the form of a single entity or administration. In other terms: the active ingredients are present in one dosage form, e.g. in one tablet, in one pre-filled syringe, or in one injection device, such as autoinjector or pen.

The invention relates to a combination therapy comprising administering a long-acting GLP-1/glucagon receptor dual agonist and a long acting NPY2 receptor agonist.

More specifically, the invention relates to a combination therapy comprising administering to a patient
an effective amount of Compound I:

(SEQ ID NO: 01, SEQ ID NO: 02)
H-His-Ac4c-Gln-Gly-Thr-Phe-Thr-Ser-Asp-Tyr-Ser-Lys-
Tyr-Leu-Asp-Glu-Arg-Ala-Ala-Lys-Asp-Phe-Ile-Lys
(HOOC—(CH₂)₁₆—CO-γGlu-Gly-Ser-Gly-Ser-Gly-Gly-)-
Trp-Leu-Glu-Ser-Ala-NH₂;

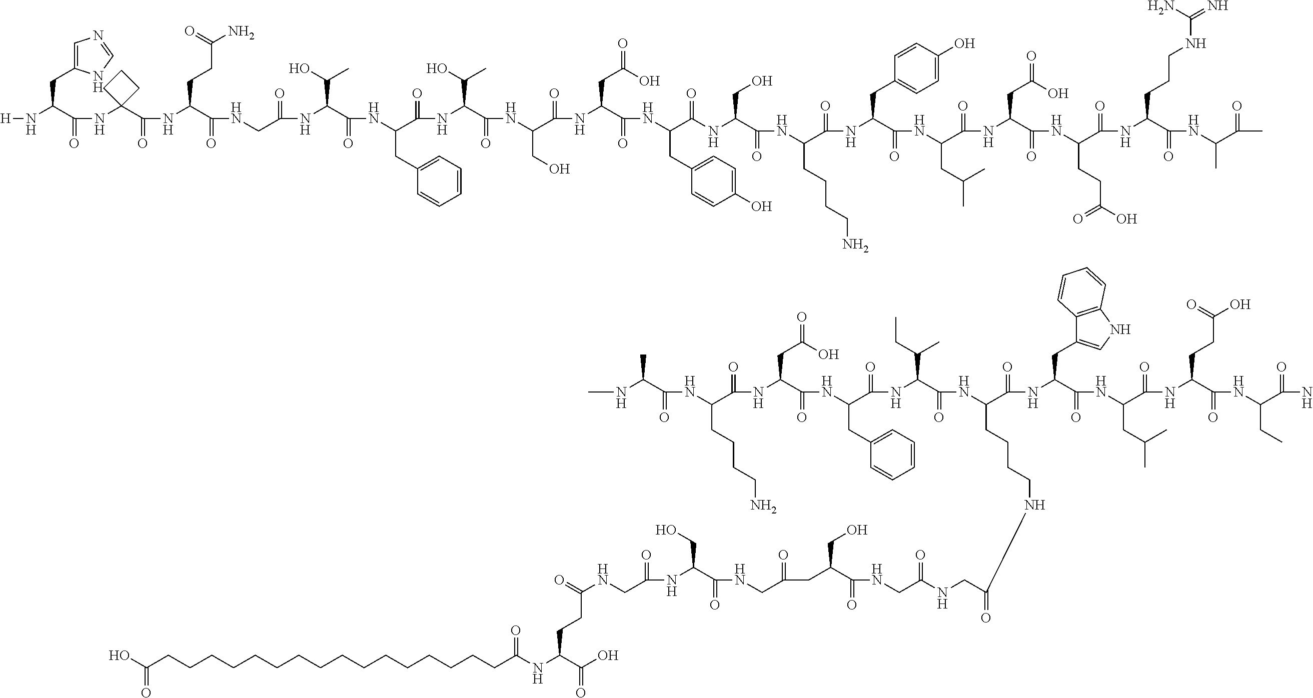

and
a long acting NPY2 receptor agonist selected from the group consisting of Compound A to L.

### Compound A

N{alpha-4}-(3-methylbutanoyl)-N{epsilon-7}-[2-(2-{2-[2-(2-{2-[(4S)-4-carboxy-4-(17- carboxyheptadecanamido)butanamido]ethoxy}ethoxy)acetamido]ethoxy}ethoxy)acetamido]-[4A,6A,7K,9E,13T,18Q,22V,28Y,30W,31L]hPYY(4-36);

(SEQ ID NO: 03)
iVal-APAK(C18DA-gGlu-OEG-OEG-)
PEEDATPEELQRYYVSLRHYYNWLTRQRY-NH₂;

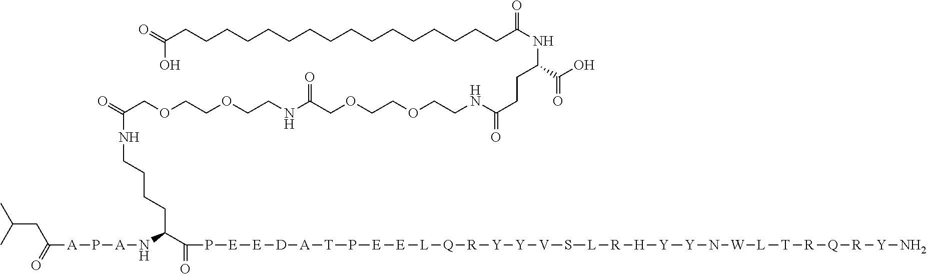

### Compound B

N{alpha-4}-(3-methylbutanoyl)-N{epsilon-7}-[2-(2-{2-[2-(2-{2-[(4S)-4-carboxy-4-(17- carboxyheptadecanamido)butanamido]ethoxy}ethoxy)acetamido]ethoxy}ethoxy)acetamido]-[4A,6A,7K,9E,18Q,22V,23A,28Y,30W,31L]hPYY(4-36);

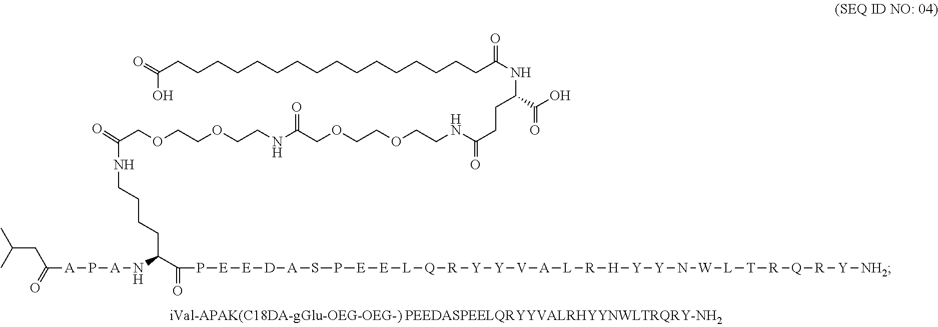

### Compound C

N{alpha-4}-(3-methylbutanoyl)-N{epsilon-7}-[2-(2-{2-[2-(2-{2-[(4S)-4-carboxy-4-(17- carboxyheptadecanamido)butanamido]ethoxy}ethoxy)acetamido]ethoxy}ethoxy)acetamido]-[4A,6A,7K,9E,11E,18Q,22V,28Y,30W,31L]hPYY(4-36);

(SEQ ID NO: 05)
iVal-APAK(C18DA-gGlu-OEG-OEG-)
PEEEASPEELQRYYVSLRHYYNWLTRQRY-NH₂;

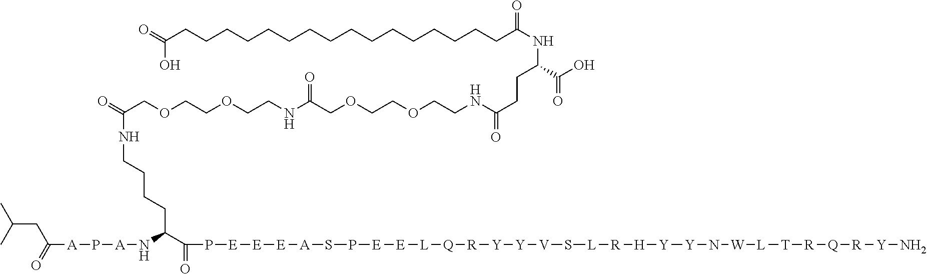

### Compound D

N{alpha-4}-(3-methylbutanoyl)-N{epsilon-7}-[2-(2-{2-[2-(2-{2-[(4S)-4-carboxy-4-(17- carboxyheptadecanamido)butanamido]ethoxy}ethoxy)acetamido]ethoxy}ethoxy)acetamido]-[4A,7K,9E,14A,18Q,22V,23A,28Y,30W,31L]hPYY(4-36);

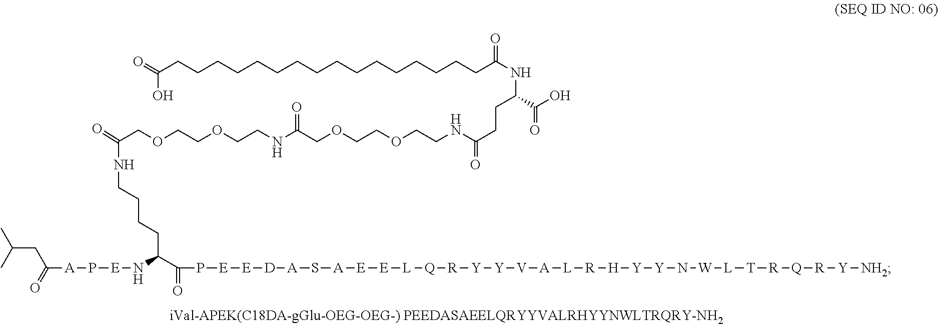

### Compound E

N{alpha-4}-(3-methylbutanoyl)-N{epsilon-7}-[2-(2-{2-[2-(2-{2-[(4S)-4-carboxy-4-(17- carboxyheptadecanamido)butanamido]ethoxy}ethoxy)acetamido]ethoxy}ethoxy)acetamido]-[4A,6A,7K,9E,17I,18Q,22V,28Y,30W,31L]hPYY(4-36);

(SEQ ID NO: 07)
iVal-APAK(C18DA-gGlu-OEG-OEG-)
PEEDASPEEIQRYYVSLRHYYNWLTRQRY-NH₂;

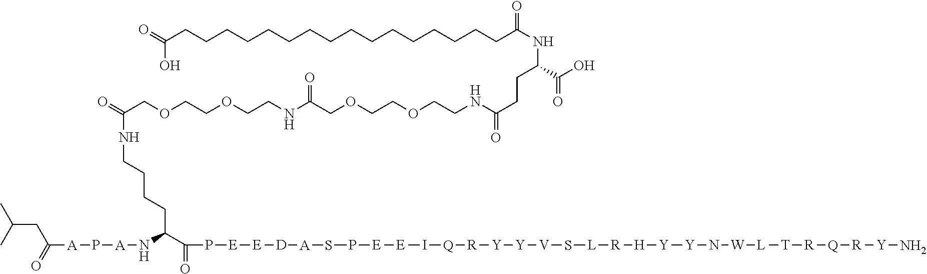

### Compound F

N{alpha-4}-(3-methylbutanoyl)-N{epsilon-7}-[2-(2-{2-[2-(2-{2-[(4S)-4-carboxy-4-(17- carboxyheptadecanamido)butanamido]ethoxy}ethoxy)acetamido]ethoxy}ethoxy)acetamido]-[4A,7K,9E,10A,13T,18Q,22V,28Y,30W,31L]hPYY(4-36);

(SEQ ID NO: 08)
iVal-APEK(C18DA-gGlu-OEG-OEG-)
PEADATPEELQRYYVSLRHYYNWLTRQRY-NH₂;

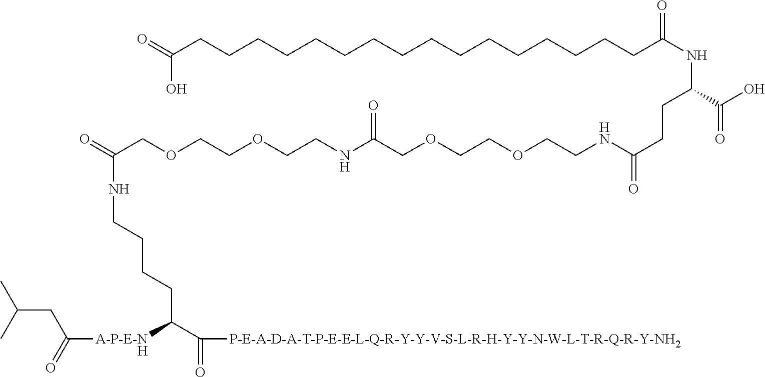

### Compound G

N{alpha-4}-(3-methylbutanoyl)-N{epsilon-7}-[2-(2-{2-[2-(2-{2-[(4S)-4-carboxy-4-(17- carboxyheptadecanamido)butanamido]ethoxy}ethoxy)acetamido]ethoxy}ethoxy)acetamido]-[4A,7K,9E,13T,17I,18Q,22V,28Y,30W,31L]hPYY(4-36);

(SEQ ID NO: 09)
iVal-APEK(C18DA-gGlu-OEG-OEG-)
PEEDATPEEIQRYYVSLRHYYNWLTRQRY-NH₂;

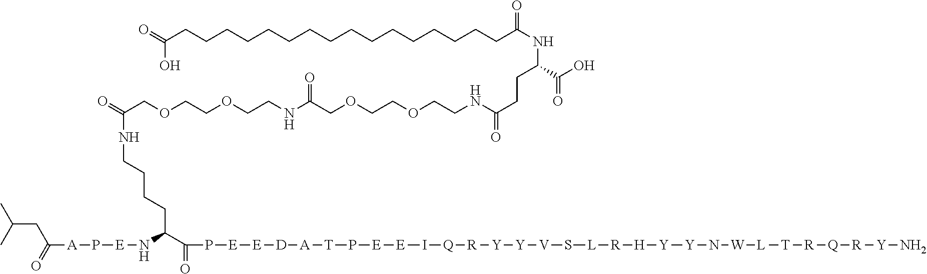

### Compound H

N{alpha-4}-(3-methylbutanoyl)-N{epsilon-7}-[2-(2-{2-[2-(2-{2-[(4S)-4-carboxy-4-(17- carboxyheptadecanamido)butanamido]ethoxy}ethoxy)acetamido]ethoxy}ethoxy)acetamido]-[4A,7K,9E,13T,18Q,19K,22V,28Y,30W,31L]hPYY(4-36);

(SEQ ID NO: 10)
iVal-APEK(C18DA-gGlu-OEG-OEG-)
PEEDATPEELQKYYVSLRHYYNWLTRQRY-NH₂;

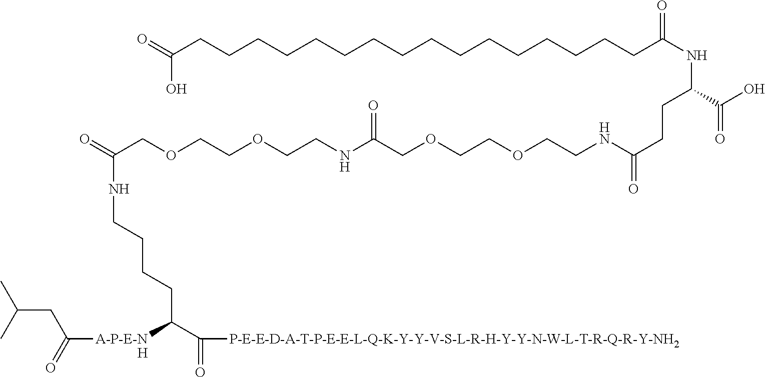

### Compound J

N{alpha-4}-(3-methylbutanoyl)-N{epsilon-7}-[2-(2-{2-[2-(2-{2-[(4S)-4-carboxy-4-(17- carboxyheptadecanamido)butanamido]ethoxy}ethoxy)acetamido]ethoxy}ethoxy)acetamido]-[4A,7K,9E,10A,17I,18Q,22V,28Y,30W,31L]hPYY(4-36);

(SEQ ID NO: 11)
iVal-APEK(C18DA-gGlu-OEG-OEG-)
PEADASPEEIQRYYVSLRHYYNWLTRQRY-NH₂;

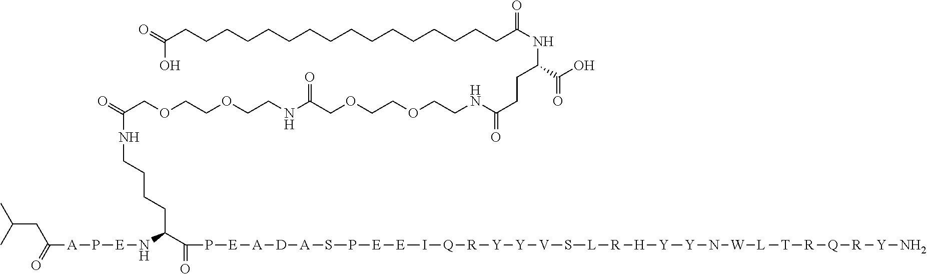

### Compound K

N{alpha-4}-(3-methylbutanoyl)-N{epsilon-7}-(6-[(4S)-4-carboxy-4-(17-carboxyheptadecanamido)butanamido]hexanoyl)-[4A,7K,9E,10A,13T,17I,18Q,22V,28Y,30W,31L]-hPYY(4-36);

(SEQ ID NO: 12)
iVal-APEK(C18DA-gGlu-Ahx)PEADATP
EEIQRYYVSLRHYYNWLTRQRY-NH₂;

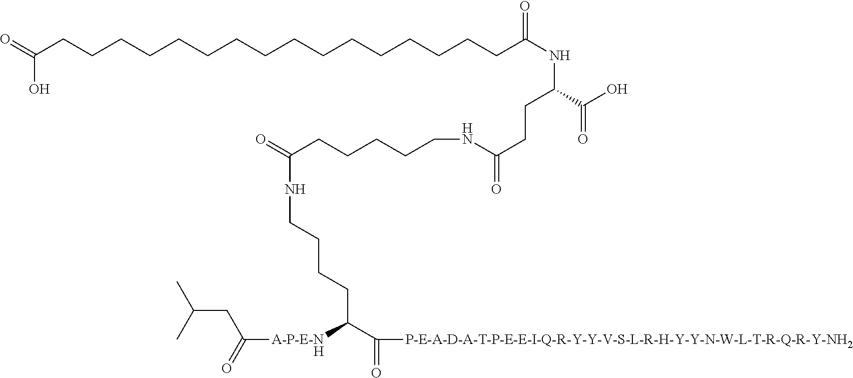

### Compound L

N{alpha-4}-(3-methylbutanoyl)-N{epsilon-7}-(6-[(4S)-4-carboxy-4-(17-carboxyheptadecanamido)butanamido]hexanoyl)-[4A,7K,9E,13T,17I,18Q,22V,23A,28Y,30W,31L]-hPYY(4-36);

(SEQ ID NO: 13)
iVal-APEK(C18DA-gGlu-Ahx)PEEDATP
EEIQRYYVALRHYYNWLTRQRY-NH₂;

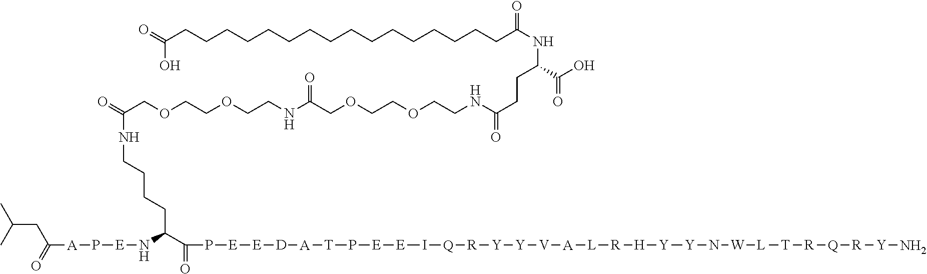

In an alternative embodiment the invention relates to a combination therapy comprising administering to a patient
an effective amount of semaglutide:

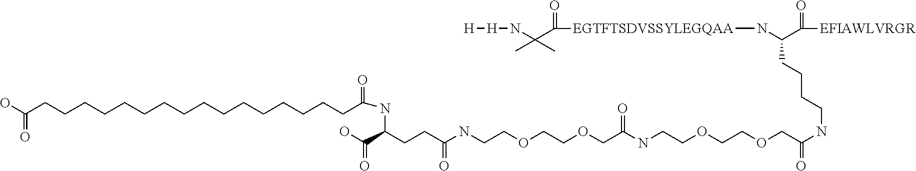

(N-epsilon26-[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Aib8, Arg34]GLP-1-(7-37), (SEQ ID NO: 17)) and
a long acting NPY2 receptor agonist selected from the group consisting of Compound A to L.

WO2021/094259, which is incorporated in its entirety by reference, discloses Ki as measured in a Radio Ligand Binding (RLB) assay (Example 1) of compounds A to J, as well as the effect on acute food intake (AFI) in normal NMRI mice (Example 6).

WO2022/029231 which is incorporated in its entirety by reference, discloses Ki as measured in a Radio Ligand Binding (RLB) assay (Example 1) of compounds K and L, as well as the effect on acute food intake (AFI) in normal NMRI mice (Example 6).

Example 1 herein describes the same Radio Ligand Binding (RLB) assay. The measured Ki for Compounds A to L and Ref. 1 to Ref. 3 are reported in Table 1 below. Example 2 herein describes an experiment to measure the effect on acute food intake (AFI) in normal NMRI mice. The AFI in % vs. the vehicle group is provided in Table 1 below. Example 3 herein describes an experiment to measure the effect on body weight loss and food intake in Diet Induced Obesity (DIO) mice.

All the compounds show good binding to the human NPY2 receptor (Example 1) and achieve a strong inhibition of food intake after 24 h (Example 2). In the DIO study the compounds A to L achieved a body weight loss in the range of 6.7% to 12.2% vs. vehicle group (Example 3). The reference compounds Ref. 1 to Ref. 3, however, achieved a body weight loss of only up to 2.7%. This shows that not all PPY2 analogues, which bind to the NPY2 receptor and which inhibit acute food intake, are in fact capable of efficaciously reduce body weight.

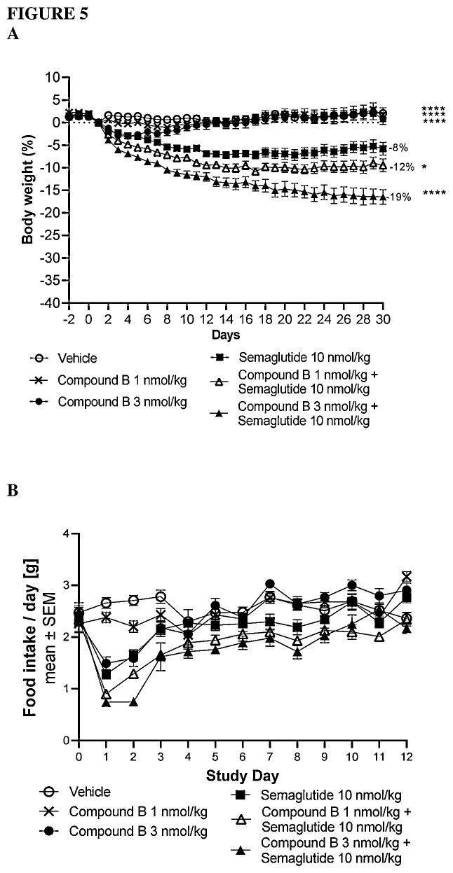

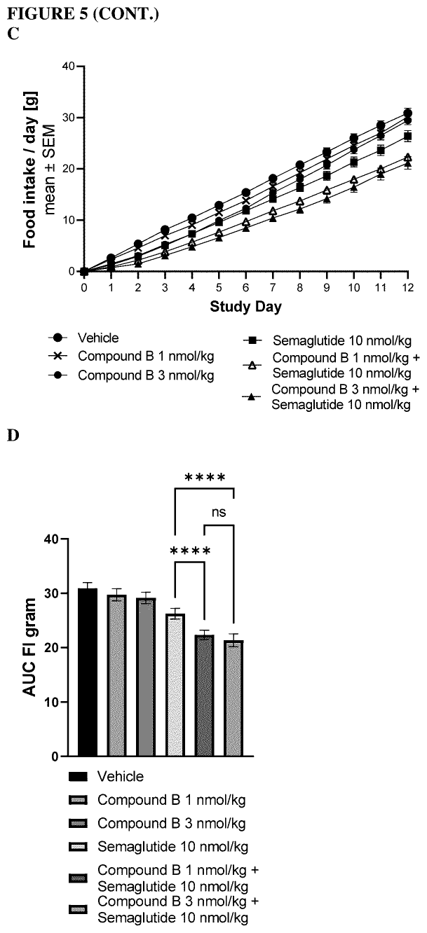

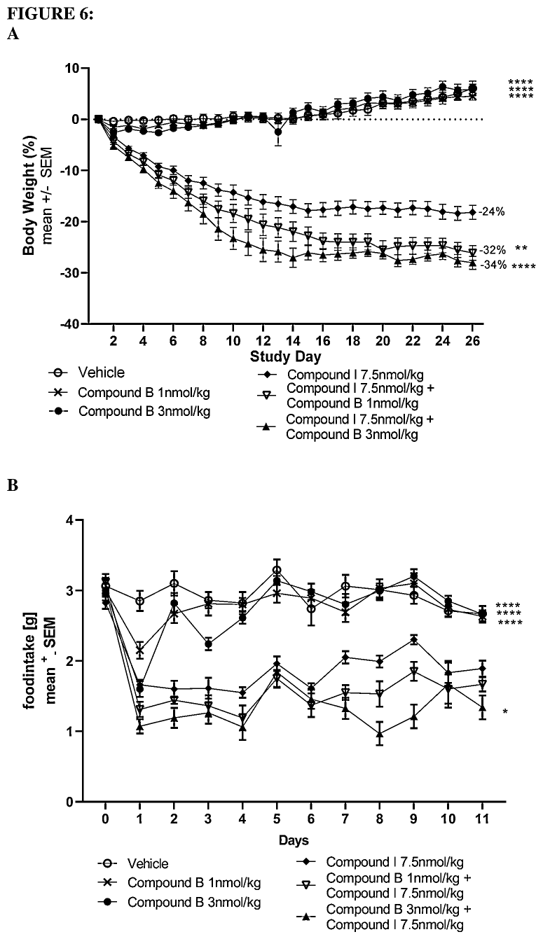

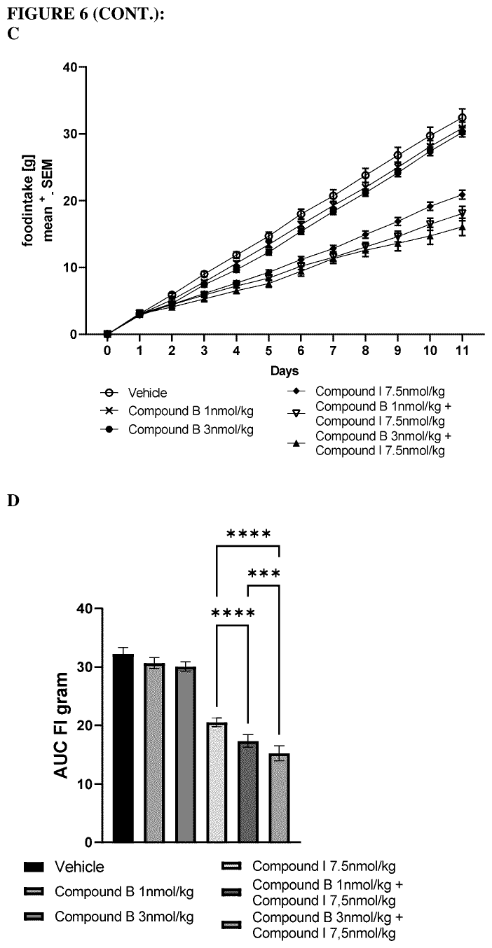

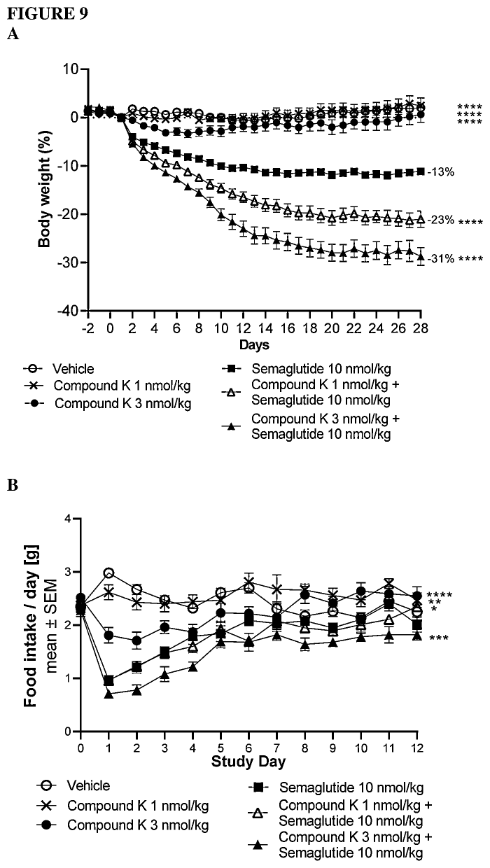

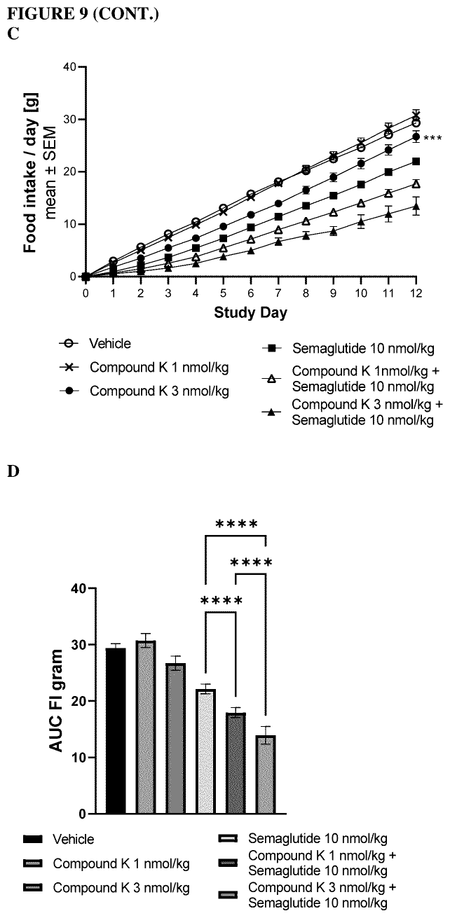

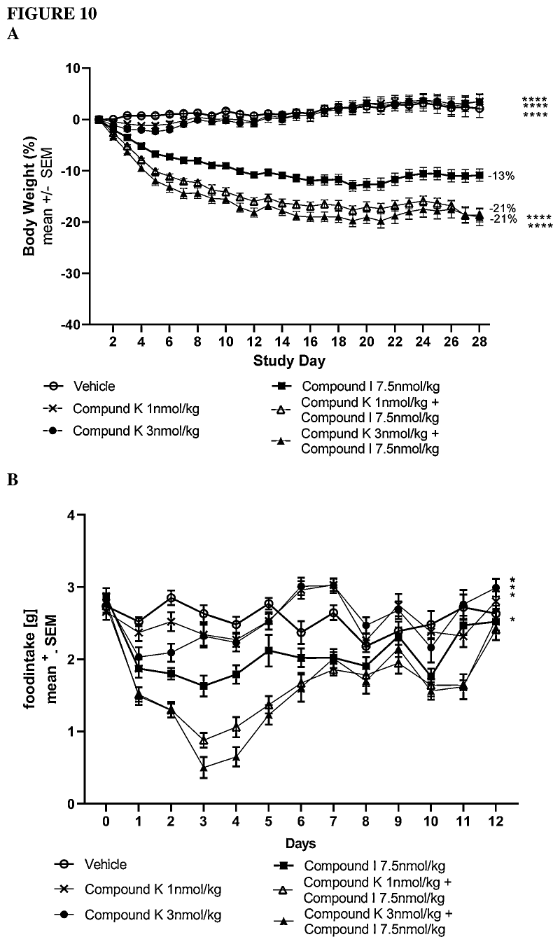

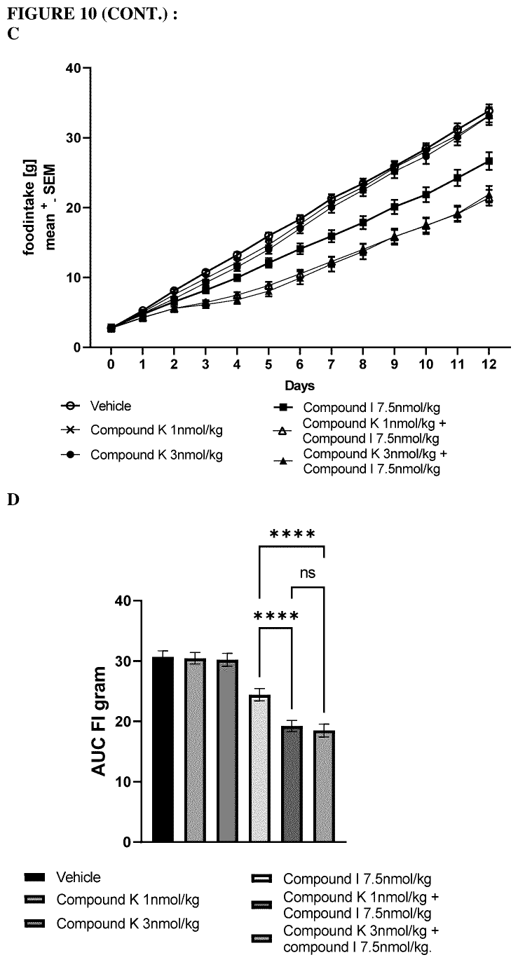

The DIO experiments (Example 3) shows that there is synergistic effect on body weight for Compounds B, E or K in combination with semaglutide (FIGS. 5A, 7A and 9A). Synergistic body weight loss is also achieved for the combination of Compounds B, E or K together with Compound I (FIGS. 6A, 8A, and 10A). A synergistic effect or greater than additive benefit may be observed when semaglutide is administered in combination with Compound B, E, or K.

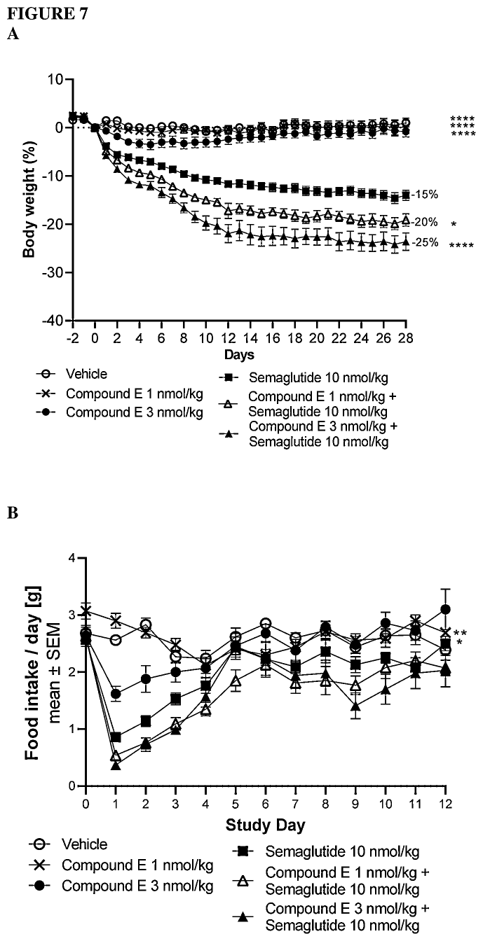

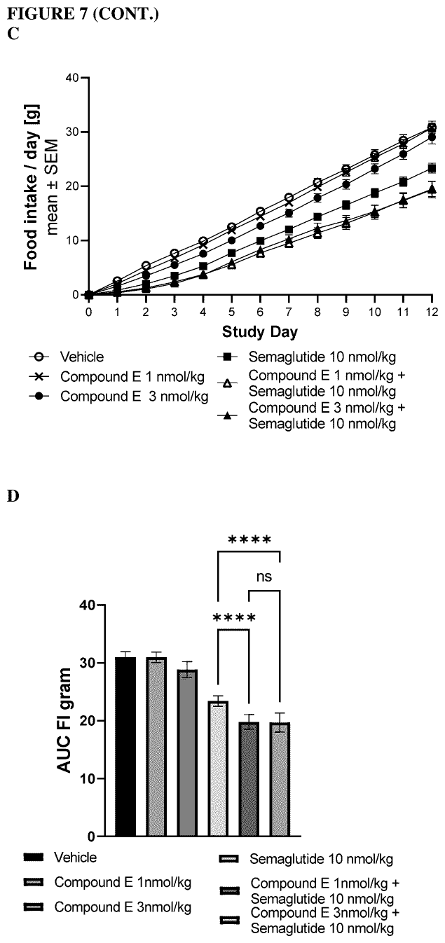

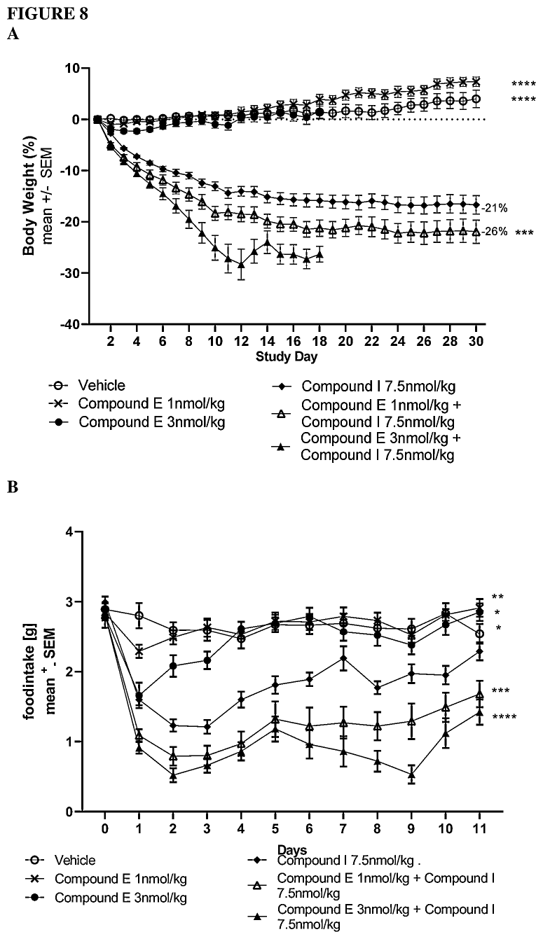

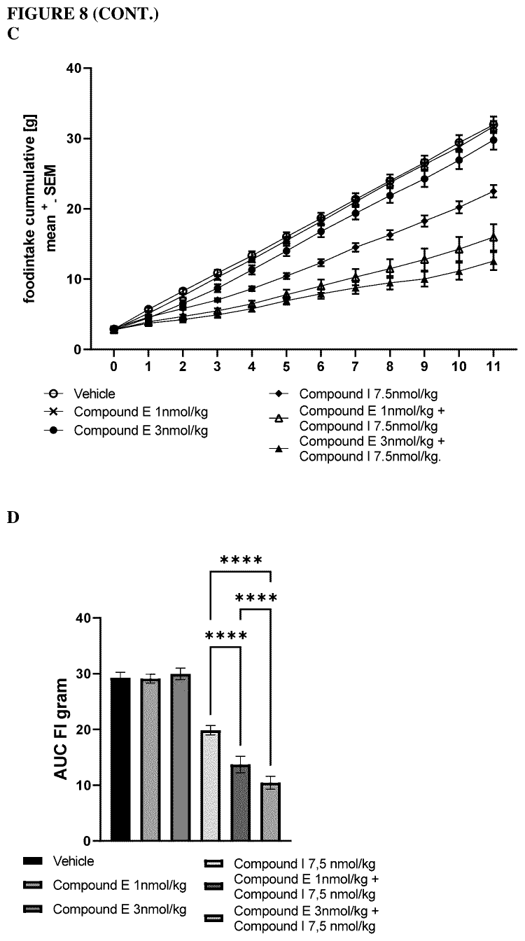

The combination of Compound B with Compound I seems to provide a long-lasting reduction on food intake (FIG. 6B) in comparison to the combination of Compound B with semaglutide (FIG. 5B). A longer-lasting effect on food intake is also observed for the combination of Compound E with Compound I (FIG. 8B) vs. Compound E and semaglutide (FIG. 7B). The long-lasting reduction in food intake for compound B and E in combination with compound I results in greater body weight lowering efficacy (% BW, FIGS. 6A and 8A) compared to the combination of compound B or E with semaglutide (FIGS. 5A and 7A), respectively, and in a pronounced total food intake reduction (FIGS. 6D and 8D).

The combination DIO experiments indicate that a synergistic effect can be achieved when combining the long-acting GLP-1/glucagon receptor dual agonist with a long acting NPY2 receptor agonist according to the invention. Further, the combinations according to the present invention might provide a longer lasting effect on pronounced food intake reduction compared to a combination comprising a long-acting GLP-1 receptor agonist (semaglutide) and NPY2 receptor agonists.

In an embodiment, the long acting NPY2 receptor agonist is selected from the group consisting of Compound B, E, K, and L.

In a further embodiment, the long acting NPY2 receptor agonist is selected from the group consisting of Compound B, E and K.

In a further embodiment, the long acting NPY2 receptor agonist is selected from the group consisting of Compound E and K.

In a further embodiment, the long acting NPY2 receptor agonist is selected from the group consisting of Compound B and E.

In a further embodiment, the long acting NPY2 receptor agonist is Compound B.

In a further embodiment, the long acting NPY2 receptor agonist is Compound E.

In a further embodiment, the long acting NPY2 receptor agonist is Compound K.

In an embodiment, the weight ratio between Compound I (or semaglutide) and the NPY2 receptor agonist is in the range of 1:10 to 10:1 (Compound I: NPY2 receptor agonist).

In an embodiment, the weight ratio between Compound I (or semaglutide) and the NPY2 receptor agonist is in the range of 1:5 to 5:1 (Compound I: NPY2 receptor agonist).

In an embodiment, the weight ratio between Compound I (or semaglutide) and the NPY2 receptor agonist is in the range of 1:3 to 3:1.

In an embodiment, the weight ratio between Compound I (or semaglutide) and the NPY2 receptor agonist is in the range of 1:2 to 2:1.

For example, the weight ratio between Compound I (or semaglutide) and Compound E is in the range of 1:3 to 3:1. Accordingly, the two compounds maybe administered in a weight ratio of 1:2, 1:1 or 2:1.

For example, the weight ratio between Compound I (or semaglutide) and Compound K is in the range of 1:3 to 3:1. Accordingly, the two compounds maybe administered in a weight ratio of 1:2, 1:1 or 2:1.

Although any combination of doses may be used, typically doses of Compound I and the NPY2R agonist that provide a synergistic effect, or greater than additive benefit, are used. For example, due to the synergistic behavior, lower doses of Compound I and the NPY2R agonist may be selected, thereby reducing the drug burden of the patient while still achieving a relevant effect on body weight. Furthermore, an acceptable tolerability and safety might be achieved providing a broad therapeutic window of such combination. Furthermore, the lower doses reduce the drug substance requirements potentially providing economic benefit.

Low doses of the compounds (e.g. Compound I and/or NPY2R agonist) may initially be administered with gradually increasing doses (dose escalation) until a certain dose (maintenance dose) is reached and maintained.

The compounds may be administered once per week (once weekly) or more often, preferably once weekly.

In an embodiment, Compound I (or semaglutide) and the long acting NPY2 receptor agonist are administered via subcutaneous injection(s), e.g. via two separate injections (“free combination”) or via one injection administering both compounds simultaneously, e.g. via injection of a fixed-dose combination of the two agonists. In case of two separate injections, one of the compounds may be administered first immediately followed by the other one, or there might be a time period in between the two injections, such as 4, 3, 2 or 1 day. Alternatively, the time period in between the injections is 12, 6, 4, 2 or 1 hour.

In a further aspect, the invention relates to the use of a PYY analogue selected from the group consisting of Compound A to L in a combination therapy together with Compound I.

Accordingly, the invention relates to a method of treatment of a human body, wherein the treatment comprises administering a (one) PYY analogue selected from the group consisting of Compound A to L and Compound I.

Accordingly, the invention relates to Compound I for use in a combination therapy together with a PYY analogue selected from the group consisting of Compound A to L.

In an alternative aspect, the invention relates to the use of a PYY analogue selected from the group consisting of Compound A to L in a combination therapy together with semaglutide.

Accordingly, the invention relates to a method of treatment of a human body, wherein the treatment comprises administering a (one) PYY analogue selected from the group consisting of Compound A to L and semaglutide.

Accordingly, the invention relates to semaglutide for use in a combination therapy together with a PYY analogue selected from the group consisting of Compound A to L.

The combination therapy of the invention may be administered in addition to a treatment with another pharmacological treatment, e.g. incretin-based therapy, as further elaborated below. In another embodiment, the combination therapy of the invention is administered without administration of further incretin-based therapies.

In a more specific embodiment, the combination therapy of the invention relates to a treatment of humans.

In an alternative embodiment, the present invention relates to a combination therapy comprising administering semaglutide and a long acting NPY2 receptor agonist selected from the group consisting of Compound A to L, preferably Compound B, E, K or L.

### Method of Treatment

The present invention is directed to a combination of PYY analogues and a dual GLP-1R/GCGR agonist according to the above-mentioned embodiments, which are useful in the prevention and/or treatment of a disease and/or condition associated with or modulated by NPY2, GLP-1, and GCG receptor activity, including but not limited to the treatment and/or prevention of obesity and various obesity-related conditions, diseases, or co-morbidities, such as type 2 diabetes, liver diseases such as NAFLD and NASH (non-alcoholic steato-hepatitis), kidney diseases, or cardiovascular diseases.

The combination therapy described herein finds use, inter alia, in preventing weight gain or promoting weight loss. By “preventing” is meant inhibiting or reducing when compared to the absence of treatment and is not necessarily meant to imply complete cessation of weight gain. The combination therapy may cause a decrease in food intake and/or increased energy expenditure and may have a beneficial effect on glucose control and/or on lipid metabolism, including but not limited to liver and circulating triglyceride and cholesterol levels, respectively and capable of lowering circulating LDL levels and increasing HDL/LDL ratio. Thus, the combination therapy of the invention can be used for direct or indirect therapy of any condition caused or characterised by excess body weight, such as the treatment and/or prevention of obesity, morbid obesity, obesity linked inflammation, obesity linked gallbladder disease, and obesity related sleep apnea. The combination therapy may also be used for the prevention of conditions or treatment of obesity associated co-comorbidities caused or characterised by inadequate glucose control or dyslipidaemia, Type 2 diabetes, metabolic syndrome, hypertension, atherogenic dyslipidemia, coronary heart, disease peripheral artery disease, stroke or microvascular disease, heart failure, and cancer. The combination may also be used for the prevention of conditions or treatment of obesity associated co-comorbidities such as liver diseases like NAFLD and NASH, kidney diseases, and treatment of diseases of the central nervous system such as cognitive dysfunction, depression, psychiatric disorders including addictive behaviours (e.g. opoid addiction, binge eating) or neurodegenerative diseases such as Alzheimer's disease or Parkinson's disease. The effects of the combination therapy in these diseases may be because of or associated with their effect on body weight or may be independent thereof.

### Combination Therapy

The combination therapy of a PYY analogue with a dual acting agonist for the GLP-1 and GCG receptor may be administered together with another active agent for the treatment of the disease or disorder in question, e.g. an anti-obesity agent, an anti-diabetic agent, an agent for treatment of metabolic syndrome, an anti-dyslipidemia agent, an anti-hypertensive agent, a proton pump inhibitor, or an anti-inflammatory agent. In such cases, is the active agents may be given together or separately, e.g. as constituents in the same pharmaceutical composition or formulation, or as separate formulations. For instance, the PYY analogue and the GLP-1/GCGR agonist may be administered together, and the additional active agent may be administered separately (e.g. as background medication).

Thus, the combination according to the invention may be used in combination with an anti-obesity agent of known type. The anti-obesity agent may be amylin or an amylin analogue, e.g. pramlintide, or an calcitonin analogue. Alternatively, the anti-obesity agent may be a lipase inhibitor (Orlistat™), phentermine, a melanin concentrating hormone receptor 1 antagonist, GDF-15 analogue, a FGF-21 analogue, a Urocortin analogue, leptin analogue, a GOAT inhibitor, a ghrelin-receptor antagonist, neuromedin receptor 2 agonists, a NPY4 receptor agonist, a NPY5 receptor antagonist, a melanocortin receptor 4 agonist, as well as analogues thereof. It will be understood that the combination of PYY analogues with dual acting agonists for the GLP-1R/GCGR may thus be administered to subjects affected by conditions or diseases characterised by inadequate control of appetite or otherwise over-feeding, such as binge-eating disorder and Prader-Willi syndrome. It will be clear that the analogues can be used for treatment of combinations of the conditions or diseases described.

Moreover, the combination according to the invention may have some benefit if administered in combination with an anti-diabetic agent of known type, e.g. selected from a SGLT2 inhibitor (i.e. an inhibitor of sodium-glucose transport, e.g. empagliflozin), a GPR40 agonist (FFAR1/FFA1 agonist), or an insulin or an insulin analogue. Examples of appropriate insulin analogues include, but are not limited to, Lantus™, Novorapid™, Humalog™, Novomix™, Actraphane™ HM, Levemir™ Degludec™ and Apidra™.

The above-mentioned invention of a combination between a PYY analogue and a dual agonist for the GLP-1R and GCGR, may further be used in combination with medications targeting cardiovascular diseases treating hypertension, dyslipidemia, inflammation and platelet function. The medication treating hypertension can be selected from the group including, but not limited to, an angiotensin-converting enzyme inhibitor, an angiotensin II receptor blocker, a diuretic, a beta-blocker or a calcium channel blocker. The medication treating heart failure can be selected from the class Angiotensin-receptor-Neprilysin-inhibitors (ARNi), SGLT2 inhibitiors (e.g. empagliflozin), soluble guanylate cyclase stimulators or activators (e.g. vericiguat), beta-blocker, an angiotensin-converting enzyme inhibitor, an angiotensin II receptor blocker, or steroidal (spironolactone) and non-steroidal (e.g. finenerenone) mineralocorticoid receptor antagonists, and aldosterone-synthase inhibitors.

The above-mentioned invention of a combination between a PYY analogue with a dual agonist for the GLP-1R and GCGR may still further be used in combination with an anti-dyslipidemia agent of known type, including, but not limited to, a statin, a fibrate, a niacin, a PSCK9 (Proprotein convertase subtilisin/kexin type 9) inhibitor, or a cholesterol absorption inhibitor.

The above-mentioned invention of a combination between a PYY analogue with a dual agonist for the GLP-1R and GCGR may also be used in combination with a proton pump inhibitor (i.e. a pharmaceutical agent possessing pharmacological activity as an inhibitor of H⁺/K⁺-ATPase) of known type, including, but not limited to, an agent of the benzimidazole derivative type or of the imidazopyridine derivative type, such as Omeprazole™. In addition, with regard to anti-inflammatory treatment, the above mentioned invention of a combination between a PYY analogue with a dual agonist for the GLP-1R and GCGR may be beneficial if administered in combination with an anti-inflammatory agent of known type, including, but not limited to: steroids and corticosteroids (e.g., prednisone, dexamethasone), non-steroidal anti-inflammatory agents (NSAIDs), such as propionic acid derivatives (e.g., ibuprofen,); acetic acid derivatives (e.g. indomethacin, diclofenac); fenamic acid derivatives (e.g. flufenamic acid, meclofenamic acid); biphenylcarboxylic acid derivatives (e.g. diflunisal and flufenisal); oxicams (e.g. isoxicam,); salicylates (e.g. acetylsalicylic acid); and pyrazolones (e.g. apazone, bezpiperylon); COX II inhibitors (e.g. rofecoxib); preparations of interferon beta (e.g. interferon beta-1a or interferon beta-1b); and certain other compounds, such as 5-aminosalicylic acid and prodrugs and pharmaceutically acceptable salts thereof.

The above-mentioned invention of a combination between a PYY analogue and a dual agonist for the GLP-1R and GCGR, may further be used in combination with medications targeting chronic kidney diseases including diabetic kidney diseases. The medication treating chronic and diabetic kidney diseases can be selected from the group including, but not limited to, an angiotensin-converting enzyme inhibitor, an angiotensin II receptor blocker, a diuretic soluble guanylate cyclase stimulators or activators, aldosterone-synthase inhibitors, and SGLT2 inhibitor (e.g. empagliflozin).

### EXAMPLES

Compound I is disclosed in WO2015/055801, which is incorporated by reference herein in its entirety, as Example 13 and has the following structure:

(SEQ ID NO: 01, SEQ ID NO: 02)
H—H-Ac4c-QGTFTSDYSKYLDERAAKDFI-K([17-carboxy-
heptadecanoyl]-isoGlu-GSGSGG)-WLESA-NH₂

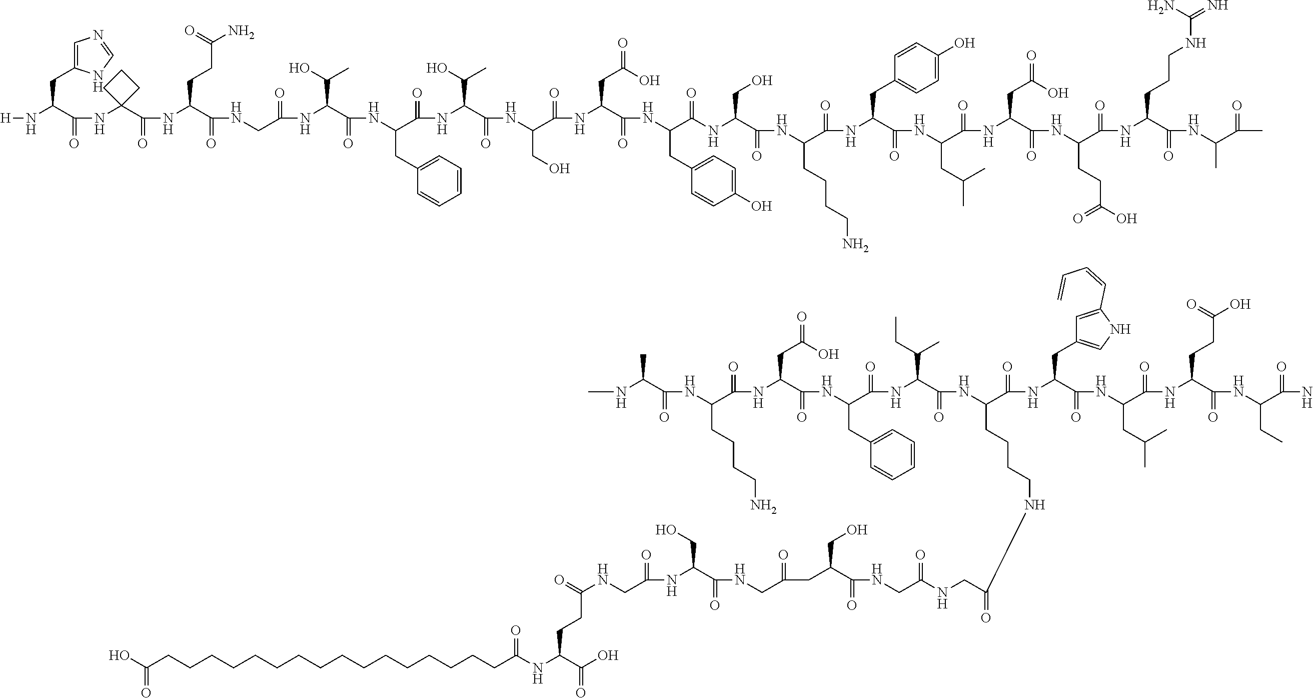

Compound A is disclosed in WO2021/094259 as Compound 106.

Compound B is disclosed in WO2021/094259 as Compound 117.

Compound C is disclosed in WO2021/094259 as Compound 234.

Compound D is disclosed in WO2021/094259 as Compound 74.

Compound E is disclosed in WO2021/094259 as Compound 23.

Compound F is disclosed in WO2021/094259 as Compound 14.

Compound G is disclosed in WO2021/094259 as Compound 171.

Compound H is disclosed in WO2021/094259 as Compound 231.

Compound J is disclosed in WO2021/094259 as Compound 53.

Compound K is disclosed in WO2022/029231 as Compound 7.

Compound L is disclosed in WO2022/029231 as Compound 84.

### Example 1

### Radioligand Binding Competition Assays (RLB)

The filtration RLB assay was carried out in 96-well plates in a final volume of 100 μl per well. Freeze-dried test peptides were dissolved in 100% dimethyl sulfoxide (DMSO) to stock solutions of 1 mM and serial dilutions were performed in assay buffer (50 mM HEPES, 5 mM MgCl₂, 1 mM CaCl₂, pH 7.4) containing 0.2% ovalbumin. 10 μl/well of the test peptide solution was added to the plates to give final concentrations ranging from 1 μM to 3 pM. Subsequently, 10 μl of human ¹²⁵I-PYY(1-36) (Perkin Elmer) in assay buffer containing 0.2% ovalbumin was added to wells to give a final concentration of 0.02 nM. Next, 80 μL membranes (HTS066M, ChemiSCREEN™ Human Neuropeptide Y2 Receptor Membrane Preparation, CHEMICON) were added to each well to give a final protein concentration of 0.5 μg/well. The plates were sealed and incubated at room temperature for 2 hours in a plate shaker set at 400 rpm. The incubation was stopped by vacuum filtration onto 0.5% poly-ethylene amine (PEI) presoaked GF/C filters using a 96-well FilterMate™ harvester (Perkin Elmer) followed by four washes with 300 μl/well ice-cold wash buffer (50 mM HEPES, 500 mM NaCl, pH7.4). Filter plates were then dried for 60 min at room temperature and the bottom of the plates was sealed with backing tape UniFilter-96. Finally, 50 μl/well scintillation counter cocktail (Microscint20, Packard) was added, and the radioactivity was counted in the Packard TopCount NXT scintillation counter. IC₅₀ values (the half maximal inhibitory concentration of the agonist) were calculated by nonlinear regression analysis of sigmoidal dose-response curves. Ki values for binding affinity were acquired by the Cheng-Prusoff equation (Ki=IC₅₀/(1+[L]/Kd), where Kd is the previously measured receptor specific dissociation constant (for NPY2R=0.07 nM) and [L] is ¹²⁵I-PYY(1-36) radioligand concentration. The Ki values are reported in Table 1 below.

### Example 2

### Effect on Acute Food Intake in Normal NMRI Mice

Male NMRI mice were obtained from Charles River (Charles River, Research Models & Services Germany GmbH) or from JanVier (JanVier Labs, France) at 5 weeks of age. The animals were group housed 4 mice pr. cage under a 12/12 h dark-light cycle, light off at 3 PM. Room temperature was controlled to 21° C.±1° C., with 60%±20% humidity. Animals had ad libitum access to regular rodent chow (KLIBA Nafag 3430 or Altromin 1324, Brogaarden, Denmark) and tap water.

Animals were transferred 5-7 days before the start of the study to a real-time food intake monitoring system, HM-2 system (MBRose, Denmark), to allow acclimatization to experimental conditions. As the animals were uniquely identified with microchips, each individual animal was identified by its microchip upon entry and exit from the food channel. Randomization of the mice for each study group (n=7-8) was based on body weight measured the day before the start of the study. A vehicle-treated (50 mM phosphate buffer pH7 with 5% Mannitol) group was included in each experiment. Six hours before the start of the night phase animals were fasted. One hour before the dark phase animals were dosed once subcutaneously (10 nmol/kg) with test peptide. Food intake was reported hourly for a period of 24 h. The food intake of the treated groups was normalized (in %) to the average food intake of the group receiving vehicle (Table 1). Statistical significance was evaluated using One-way analysis of variance with Turkey's multiple comparison test. P<0.05 was considered statistically significant.

### Example 3

### Effect on Body Weight Loss in Wildtype Diet Induced Obesity Mice

Male C57BL6/J pre-fed on 60% high fat diet were obtained from The Jackson Laboratories at >16 weeks of age. Upon arrival of the Diet Induced Obesity (DIO) mice, the mice where single housed to obtain accurate and individual food intake measurements of each animal and assigned an animal number. Animals were housed at a room temperature of 21±2° C., relative humidity 60±20% and a reversed 12-h light/dark cycle (lights of at 10 am). During the entire study animals had ad libitum access to food and water.

Before start of treatment a stratified randomization was performed based on the body weight measured in week −1 prior study start. At study start the age of mice was around 20 weeks. Bodyweight and food intake were measured daily before compound administration. Animals were dosed around one hour before start of the night phase by subcutaneous injection of 30 nmol/kg once daily for 5 till 28 days depending on the experiment. Control animals were dosed by subcutaneous injection daily with vehicle.

### RLB Assay (NPY2R)

TABLE 1

| | hY2 | AFI 10 | BW 30 |
|---|---|---|---|
| | RLB | nmol/kg | nmol/kg |
| Compound # | (nM) | 24 h (%) | 5 day (%) |
| Ref 1 | 0.74 | 42 | 1.9 |
| Ref 2 | 0.40 | 32 | 2.7 |
| Ref 3 | 0.46 | 36 | 2.7 |
| A | 0.66 | 39 | 6.7 |
| B | 0.53 | 44 | 6.7 |
| C | 1.95 | 42 | 6.9 |
| D | 0.57 | 41 | 7.0 |
| E | 0.55 | 48 | 7.3 |
| F | 0.31 | 46 | 7.3 |
| G | 0.34 | 44 | 10.6 |
| H | 1.96 | 39 | 10.6 |
| J | 0.24 | 29 | 12.2 |
| K | 1.04 | | 8.0 |
| L | 1.60 | | 7.3 |

### Reference 1

(SEQ ID NO: 14)
iVal-APEK(C18DA-gGlu-OEG-OEG)PEEDASPEEIQQYYVSLRHYY
NWLTRQRY-NH₂

Reference 1 is disclosed in WO2021/094259 as Compound 11.

### Reference 2

(SEQ ID NO: 15)
iVal-APEK(C18DA-gGlu-OEG-OEG)PGEDASPEELQRYYVSLRHYY
HWLTRQRY-NH₂

### Reference 3

(SEQ ID NO: 16)
iVal-RPEK(C18DA-gGlu-OEG-OEG)PEEDASPEELQRYYVSLRHYY
NWLTRQRY-NH₂

What we claim:

1. A method for treatment and/or prevention of obesity and/or an obesity-related condition, comprising treating a patient in need thereof with a combination therapy comprising administering a long-acting GLP-1/glucagon receptor dual agonist and a long acting NPY2 receptor agonist.

2. The method according to claim 1 wherein the long-acting GLP-1/glucagon receptor dual agonist is:
H-His-Ac4c-Gln-Gly-Thr-Phe-Thr-Ser-Asp-Tyr-Ser-Lys-Tyr-Leu-Asp-Glu-Arg-Ala-Ala-Lys-Asp-Phe-Ile-Lys(HOOC—(CH₂)₁₆-CO-γGlu-Gly-Ser-Gly-Ser-Gly-Gly-)-Trp-Leu-Glu-Ser-Ala-NH₂(SEQ ID NO: 01, SEQ ID NO: 02) and
wherein the long acting NPY2 receptor agonist is selected from the group consisting of Compound A, B, C, D, E, F, G, H, I, J, K and L.

3. The method according to claim 2, wherein the long acting NPY2 receptor agonist is selected from the group consisting of Compound B, E, K, and L.

4. The method according to claim 2, wherein the long acting NPY2 receptor agonist is selected from the group consisting of Compound B, E and K.

5. The method according to claim 2, wherein the long acting NPY2 receptor agonist is Compound B.

6. The method according to claim 2, wherein the long acting NPY2 receptor agonist is Compound E.

7. The method according to claim 2, wherein the long acting NPY2 receptor agonist is Compound K.

8. The method according to claim 1, wherein the combination therapy is administered once per week.

9. The method according to claim 1, wherein the obesity-related condition comprises one or more of type 2 diabetes, liver disease, kidney disease, or cardiovascular disease.

10. The method of claim 9, wherein the liver disease is NAFLD (non-alcoholic fatty liver disease) or NASH (non-alcoholic steatohepatitis).

11. The method according to claim 1, wherein the combination therapy has a synergistic effect.

12. The method according to claim 11, wherein the synergistic effect is a greater than additive improvement in therapeutic efficacy as measured by one or more of reduction of food intake, reduction of appetite and/or reduction of body weight.

13. The method according to claim 1, wherein the combination therapy is administered in a therapeutically effective amount.

14. A pharmaceutical composition comprising a combination of Compound I:
H-His-Ac4c-Gln-Gly-Thr-Phe-Thr-Ser-Asp-Tyr-Ser-Lys-Tyr-Leu-Asp-Glu-Arg-Ala-Ala-Lys-Asp-Phe-Ile-Lys(HOOC—(CH₂)₁₆-CO-γGlu-Gly-Ser-Gly-Ser-Gly-Gly-)-Trp-Leu-Glu-Ser-Ala-NH₂ (SEQ ID NO:1); and
a long acting NPY2 receptor agonist selected from the group consisting of Compound A, B, C, D, E, F, G, H, I, J, K and L.

15. The pharmaceutical composition of claim 14, wherein combination is in a therapeutically effective amount.

16. The pharmaceutical composition of claim 14, wherein the long acting NPY2 receptor agonist is selected from the group consisting of Compound B, E, K, and L.

17. The pharmaceutical composition of claim 14, wherein the long acting NPY2 receptor agonist is selected from the group consisting of Compound B, E and K.

18. The pharmaceutical composition of claim 14, wherein the long acting NPY2 receptor agonist is Compound B.

19. The pharmaceutical composition of claim 14, wherein the long acting NPY2 receptor agonist is Compound E.

20. The pharmaceutical composition of claim 14, wherein the long acting NPY2 receptor agonist is Compound K.

21. The pharmaceutical composition of claim 14, wherein the combination has a synergistic effect.

22. The pharmaceutical composition of claim 21, wherein the synergistic effect is a greater than additive improvement in therapeutic efficacy as measured by one or more of reduction of food intake, reduction of appetite and/or reduction of body weight.


SEQUENCE LISTING (16 sequences)

SEQ ID NO: 1 - protein, 29 aa, synthetic construct
  REGION 1..29: note = glucagon analogue
  SITE 2: note = 1-amino-1-cyclobutane carboxylic acid
  SITE 24: note = The epsilon amino group of Lys is linked to Gly6 of SEQ ID No: 2
  MOD_RES 29: note = C-terminal amidation
HXQGTFTSDYSKYLDERAAKDFIKWLESA

SEQ ID NO: 2 - protein, 7 aa, synthetic construct
  SITE 1: note = iso-glutamine; the alpha amino group of iso-glutamine is substituted with [17-carboxy-heptadecanoyl]
  SITE 7: note = Gly is linked to the epsilon amino group of Lys24 of SEQ ID No: 1
XGSGSGG

SEQ ID NO: 3 - protein, 33 aa, synthetic construct
  MOD_RES 33: note = C-terminal amidation
  MOD_RES 1: note = N-term is substituted with 3-methylbutanoyl
  SITE 4: note = The epsilon amino group of Lys has the following substituent: 2-(2-{2-[2-(2-{2-[(4S)-4-carboxy-4-(17-carbox yheptadecanamido)butanamido]ethoxy}ethoxy)acetamido]ethoxy }ethoxy)acetyl
APAKPEEDATPEELQRYYVSLRHYYNWLTRQRY

SEQ ID NO: 4 - protein, 33 aa, synthetic construct
  MOD_RES 33: note = C-terminal amidation
  MOD_RES 1: note = N-term is substituted with 3-methylbutanoyl
  SITE 4: note = The epsilon amino group of Lys has the following substituent:2-(2-{2-[2-(2-{2-[(4S)-4-carboxy-4-(17-carboxy heptadecanamido)butanamido]ethoxy}ethoxy)acetamido]ethoxy} ethoxy)acetyl
APAKPEEDASPEELQRYYVALRHYYNWLTRQRY

SEQ ID NO: 5 - protein, 33 aa, synthetic construct
  MOD_RES 33: note = C-terminal amidation
  MOD_RES 1: note = N-term is substituted with 3-methylbutanoyl
  SITE 4: note = The epsilon amino group of Lys has the following substituent:2-(2-{2-[2-(2-{2-[(4S)-4-carboxy-4-(17-carboxy heptadecanamido)butanamido]ethoxy}ethoxy)acetamido]ethoxy} ethoxy)acetyl
APAKPEEEASPEELQRYYVSLRHYYNWLTRQRY

SEQ ID NO: 6 - protein, 33 aa, synthetic construct
  MOD_RES 33: note = C-terminal amidation
  MOD_RES 1: note = N-term is substituted with 3-methylbutanoyl
  SITE 4: note = The epsilon amino group of Lys has the following substituent:2-(2-{2-[2-(2-{2-[(4S)-4-carboxy-4-(17-carboxy heptadecanamido)butanamido]ethoxy}ethoxy)acetamido]ethoxy} ethoxy)acetyl
APEKPEEDASAEELQRYYVALRHYYNWLTRQRY

SEQ ID NO: 7 - protein, 33 aa, synthetic construct
  MOD_RES 33: note = C-terminal amidation
  MOD_RES 1: note = N-term is substituted with 3-methylbutanoyl
  SITE 4: note = The epsilon amino group of Lys has the following substituent:2-(2-{2-[2-(2-{2-[(4S)-4-carboxy-4-(17-carboxy heptadecanamido)butanamido]ethoxy}ethoxy)acetamido]ethoxy} ethoxy)acetyl
APAKPEEDASPEEIQRYYVSLRHYYNWLTRQRY

SEQ ID NO: 8 - protein, 33 aa, synthetic construct
  MOD_RES 33: note = C-terminal amidation
  MOD_RES 1: note = N-term is substituted with 3-methylbutanoyl
  SITE 4: note = The epsilon amino group of Lys has the following substituent:2-(2-{2-[2-(2-{2-[(4S)-4-carboxy-4-(17-carboxy heptadecanamido)butanamido]ethoxy}ethoxy)acetamido]ethoxy} ethoxy)acetyl
APEKPEADATPEELQRYYVSLRHYYNWLTRQRY

SEQ ID NO: 9 - protein, 33 aa, synthetic construct
  MOD_RES 33: note = C-terminal amidation
  MOD_RES 1: note = N-term is substituted with 3-methylbutanoyl
  SITE 4: note = The epsilon amino group of Lys has the following substituent:2-(2-{2-[2-(2-{2-[(4S)-4-carboxy-4-(17-carboxy heptadecanamido)butanamido]ethoxy}ethoxy)acetamido]ethoxy} ethoxy)acetyl
APEKPEEDATPEEIQRYYVSLRHYYNWLTRQRY

SEQ ID NO: 10 - protein, 33 aa, synthetic construct
  MOD_RES 33: note = C-terminal amidation
  MOD_RES 1: note = N-term is substituted with 3-methylbutanoyl
  SITE 4: note = The epsilon amino group of Lys has the following substituent:2-(2-{2-[2-(2-{2-[(4S)-4-carboxy-4-(17-carboxy heptadecanamido)butanamido]ethoxy}ethoxy)acetamido]ethoxy} ethoxy)acetyl
APEKPEEDATPEELQKYYVSLRHYYNWLTRQRY

SEQ ID NO: 11 - protein, 33 aa, synthetic construct
  MOD_RES 33: note = C-terminal amidation
  MOD_RES 1: note = N-term is substituted with 3-methylbutanoyl
  SITE 4: note = The epsilon amino group of Lys has the following substituent:2-(2-{2-[2-(2-{2-[(4S)-4-carboxy-4-(17-carboxy heptadecanamido)butanamido]ethoxy}ethoxy)acetamido]ethoxy} ethoxy)acetyl
APEKPEADASPEEIQRYYVSLRHYYNWLTRQRY

SEQ ID NO: 12 - protein, 33 aa, synthetic construct
  MOD_RES 33: note = C-terminal amidation
  MOD_RES 1: note = N-term is substituted with 3-methylbutanoyl
  SITE 4: note = The epsilon amino group of Lys has the following substituent: 6-((4S)-4-carboxy-4-(17-carboxyheptadecanamid o)butanamido)hexanoyl
APEKPEADATPEEIQRYYVSLRHYYNWLTRQRY

SEQ ID NO: 13 - protein, 33 aa, synthetic construct
  MOD_RES 33: note = C-terminal amidation
  MOD_RES 1: note = N-term is substituted with 3-methylbutanoyl
  SITE 4: note = The epsilon amino group of Lys has the following substituent: 6-((4S)-4-carboxy-4-(17-carboxyheptadecanamid o)butanamido]hexanoyl
APEKPEEDATPEEIQRYYVALRHYYNWLTRQRY

SEQ ID NO: 14 - protein, 33 aa, synthetic construct
  MOD_RES 33: note = C-terminal amidation
  MOD_RES 1: note = N-term is substituted with 3-methylbutanoyl
  SITE 4: note = The epsilon amino group of Lys has the following substituent:2-(2-{2-[2-(2-{2-[(4S)-4-carboxy-4-(17-carboxy heptadecanamido)butanamido]ethoxy}ethoxy)acetamido]ethoxy} ethoxy)acetyl
APEKPEEDASPEEIQQYYVSLRHYYNWLTRQRY

SEQ ID NO: 15 - protein, 33 aa, synthetic construct
  MOD_RES 33: note = C-terminal amidation
  MOD_RES 1: note = N-term is substituted with 3-methylbutanoyl
  SITE 4: note = The epsilon amino group of Lys has the following substituent:2-(2-{2-[2-(2-{2-[(4S)-4-carboxy-4-(17-carboxy heptadecanamido)butanamido]ethoxy}ethoxy)acetamido]ethoxy} ethoxy)acetyl
APEKPGEDASPEELQRYYVSLRHYYHWLTRQRY

SEQ ID NO: 16 - protein, 33 aa, synthetic construct
  MOD_RES 33: note = C-terminal amidation
  SITE 4: note = The epsilon amino group of Lys has the following substituent:2-(2-{2-[2-(2-{2-[(4S)-4-carboxy-4-(17-carboxy heptadecanamido)butanamido]ethoxy}ethoxy)acetamido]ethoxy} ethoxy)acetyl
  MOD_RES 1: note = N-term is substituted with 3-methylbutanoyl
RPEKPEEDASPEELQRYYVSLRHYYNWLTRQRY